(19) United States
(12) Patent Application Publication
(10) Pub. No.: US 2016/0220234 A1
(43) Pub. Date: Aug. 4, 2016

(54) MULTIPLE SAMPLE BIOPSY DEVICE

(71) Applicant: Boston Scientific Scimed, Inc., Maple Grove, MN (US)

(72) Inventors: Devon N. Arnholt, Shoreview, MN (US); Jonathan P. FETTIG, Blaine, MN (US)

(73) Assignee: Boston Scientific Scimed, Inc., Maple Grove, MN (US)

(21) Appl. No.: 15/012,077

(22) Filed: Feb. 1, 2016

Related U.S. Application Data
(60) Provisional application No. 62/111,821, filed on Feb. 4, 2015.

(51) Int. Cl. A61B 10/02

(52) U.S. Cl. CPC A61B 10/0275; A61B 2217/007; A61B 2010/0208

(57) ABSTRACT

A device for acquiring a tissue sample includes an outer sheath extending longitudinally from a proximal end to a distal end and including a lumen extending therethrough; an inner needle extending longitudinally from a proximal end to a distal end and sized and shaped to be slidably received within the lumen of the outer sheath to be moved between an insertion configuration and a tissue collecting configuration; and a handle assembly connected to a proximal end of the outer sheath. The handle assembly includes a first port configured to be connected to a fluid flushing source and a second port configured to be connected to a collection element so that, when the inner needle is drawn proximally relative to the outer sheath to a sample collection configuration, the sample is flushable into the collection element.

### CROSS-REFERENCE TO RELATED APPLICATIONS

This application claims the benefit of U.S. Provisional Application No. 62/111,821, filed Feb. 4, 2015, the disclosure of which is incorporated herein by reference in its entirety.

### BACKGROUND OF THE DISCLOSURE

Needle biopsy procedures are common for the diagnosis and the staging of disease. For example, a percutaneous biopsy needle may be introduced through an incision in the skin to a target tissue site. In certain clinical situations it may be difficult to acquire a suitable sample via a single pass of the needle to the target site. The more cells or tissue that can be acquired, the greater the potential for a definitive diagnosis. Multiple insertions of the biopsy needle, however, may increase a risk of infection and/or seeding of healthy tissue with unhealthy tissue.

### SUMMARY OF THE DISCLOSURE

The present disclosure is directed to a device for acquiring a tissue sample, comprising an outer sheath extending longitudinally from a proximal end to a distal end and including a lumen extending therethrough along with an inner needle extending longitudinally from a proximal end to a distal end and sized and shaped to be slidably received within the lumen of the outer sheath to be moved between an insertion configuration and a tissue collecting configuration and a handle assembly connected to a proximal end of the outer sheath and including a first port configured to be connected to a fluid flushing source and a second port configured to be connected to a collection element so that, when the inner needle is drawn proximally relative to the outer sheath to the tissue collection configuration, the sample is flushable into the collection element.

In an embodiment, the needle includes a notch extending laterally therein along a distal portion thereof so that, in the insertion configuration the notch is proximal of the distal end of the outer sheath and in the tissue collecting configuration the notch is distal of the distal end of the outer sheath

In an embodiment, the handle assembly may include a first handle member connected to the proximal end of the outer sheath and a second handle member connected to the proximal end of the inner needle, the first handle member including a handle lumen through which a portion of the second handle is slidably received.

In an embodiment, the first and second handle members may be longitudinally movable relative to one another to move the device between the insertion configuration and the tissue collection configuration.

In an embodiment, the first and second ports may be diametrically opposed from one another along a housing of the first handle member so that in the sample collection configuration the notch of the needle is aligned between the first and second ports.

In an embodiment, the first and second handle members may be rotatable relative to one another about a longitudinal axis of the device to move the device between a locked configuration, in which the needle and the outer sheath are not movable relative to one another between the insertion and sample collection configurations, and an unlocked configuration in which the needle and the outer sheath are movable relative to one another between the insertion and sample collection configurations.

In an embodiment, the first and second handle members may be keyed relative to one another and include a corresponding groove and pin.

In an embodiment, one of the inner sleeve and the outer sheath may extend along a substantially straight line.

In an embodiment, one of the inner sleeve and the outer sheath may extend along a curve.

In an embodiment, the handle assembly may include a spring biasing the device toward the insertion configuration.

The present disclosure is also directed to a biopsy needle, comprising an outer sheath extending longitudinally from a proximal end to a distal end and including a lumen extending therethrough along with an inner needle extending longitudinally from a proximal end to a distal end and sized and shaped to be slidably received within the lumen of the outer sheath to be moved between an insertion configuration and a tissue collecting configuration, the needle including a notch extending laterally therein along a distal portion thereof, in the insertion configuration the notch is proximal of the distal end of the outer sheath and in the tissue collecting configuration the notch is distal of the distal end of the outer sheath and a handle assembly including a first handle member connected to a proximal end of the outer sheath and a second handle member connected to the proximal end of the inner needle, the first and second handle members longitudinally movable relative to one another to move the inner needle and the outer needle relative to one another between the insertion configuration and the tissue collection configuration, the first handle including a first port configured to be connected to a fluid flushing source and a second port configured to be connected to a tissue collection element so that, when the tissue sample is received within the notch and the inner needle is drawn proximally relative to the outer sheath to a sample collection configuration, the tissue sample is flushable from the notch into the tissue collection element.

In an embodiment, the handle assembly may include a spring element biasing the inner needle and outer sleeve toward the insertion configuration.

In an embodiment, the first and second handle members may be rotatable relative to one another about a longitudinal axis of the device to move the device between a locked configuration, in which the needle and the outer sheath are not movable relative to one another between the insertion and sample collection configurations, and an unlocked configuration in which the needle and the outer sheath are movable relative to one another between the insertion and sample collection configurations.

In an embodiment, the first and second handle members may be keyed relative to one another and include a corresponding groove and pin

In an embodiment, the first and second ports may be diametrically opposed from one another along a housing of the first handle member so that in the sample collection configuration the notch of the needle is aligned between the first and second ports.

In an embodiment, one of the inner needle and the outer sheath may extend along a curve.

The present disclosure is also directed to a method for acquiring a tissue sample, comprising inserting a distal portion of a biopsy device to a target tissue site within a patient body via a skin incision, the distal portion of the biopsy device including an outer sheath and an inner needle slidably received therein, the inner needle including a notch extending laterally therein along a distal portion thereof, moving the device from the insertion configuration, in which the notch of the inner needle is proximal of a distal end of the outer sheath, to a tissue collecting configuration in which the notch extends distally past the distal end of the outer sheath to collect a first tissue sample therein, advancing the outer sheath distally over the notch of the inner needle back to the insertion configuration to capture the first tissue sample in the notch, moving the inner needle proximally relative to the outer sheath until the notch is aligned between a first port and a second port of a handle assembly connected to a proximal end of the outer sheath, and flushing the first tissue sample from the notch by passing a fluid through the first port into the notch and collecting the first tissue sample in a tissue storage element connected to the second port.

### BRIEF DESCRIPTION OF THE DRAWINGS

The accompanying drawings, which are incorporated in and constitute a part of this specification, illustrate various exemplary aspects and together with the description, serve to explain the principles of the disclosed examples.

FIG. 1 shows a perspective view of a device according to an exemplary embodiment of the present disclosure;

FIG. 2 shows a partial longitudinal cross-sectional view of a distal portion of the device of FIG. 1;

FIG. 3 shows a longitudinal side view of a curved outer sheath of the device of FIG. 1;

FIG. 4 shows a longitudinal side view of a curved inner needle of the device of FIG. 1;

FIG. 5 shows a perspective view of a handle assembly of the device of FIG. 1;

FIG. 6 shows a side view of the handle assembly of FIG. 5, in a locked configuration; and

FIG. 7 shows a side view of the handle assembly of FIG. 5, in an unlocked configuration.

### DETAILED DESCRIPTION

The present disclosure may be further understood with reference to the following description and the appended drawings, wherein like elements are referred to with the same reference numerals. The present disclosure is directed to a biopsy device. Exemplary embodiments of the present disclosure describe a biopsy device comprising an outer sheath through which an inner sampling needle may be slidably received so that the inner needle may be removed from and inserted into the target site via the outer sheath, as desired, to collect multiple tissue samples from the target site. A tissue sample may be flushed from the inner needle and collected within a collection vial connected to a handle housing of the device so that the inner needle may be reinserted to the target site to collect additional tissue samples. It should be noted that the terms “proximal” and “distal” are intended to refer to a direction toward (proximal) and away from (distal) a user of the device.

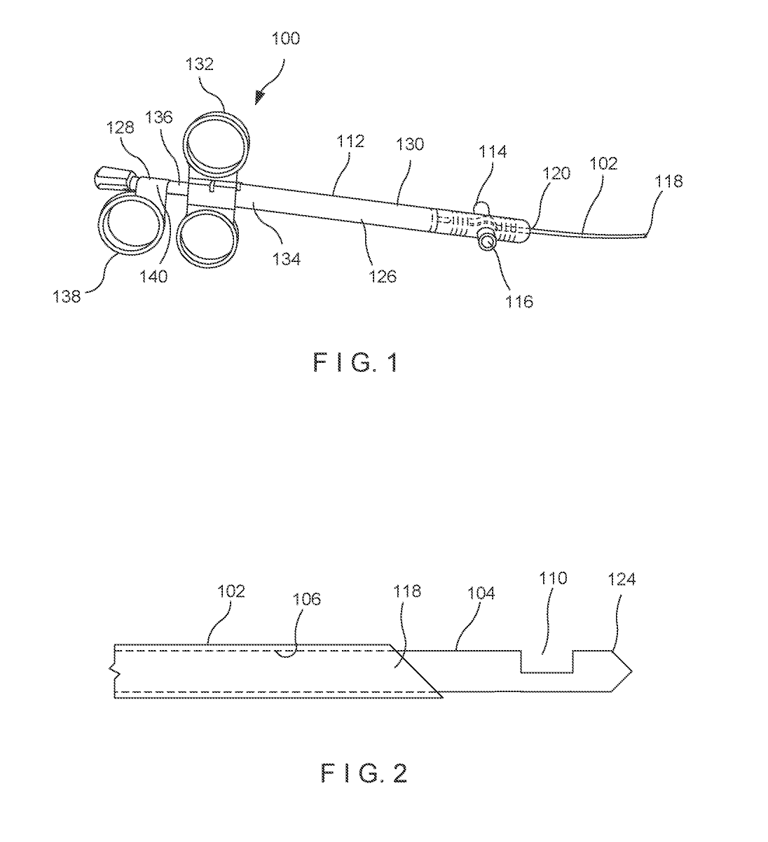

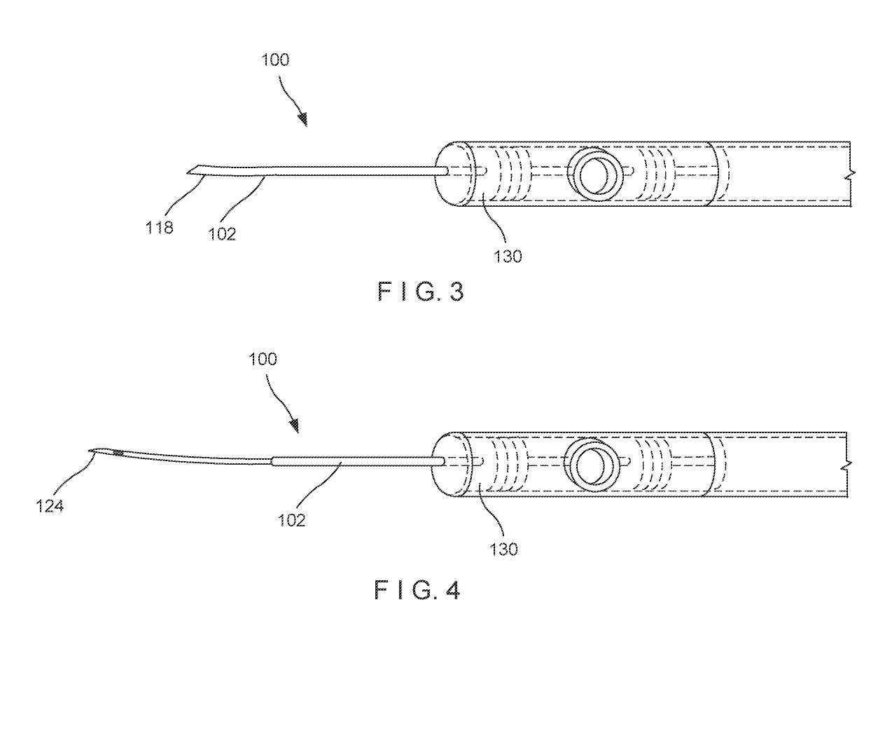

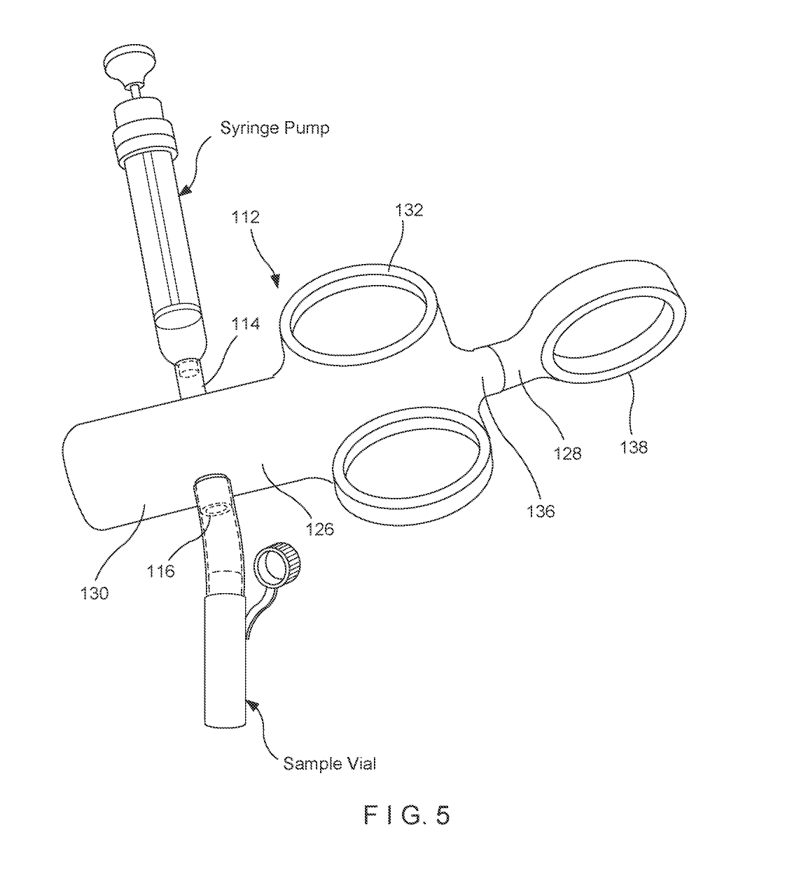

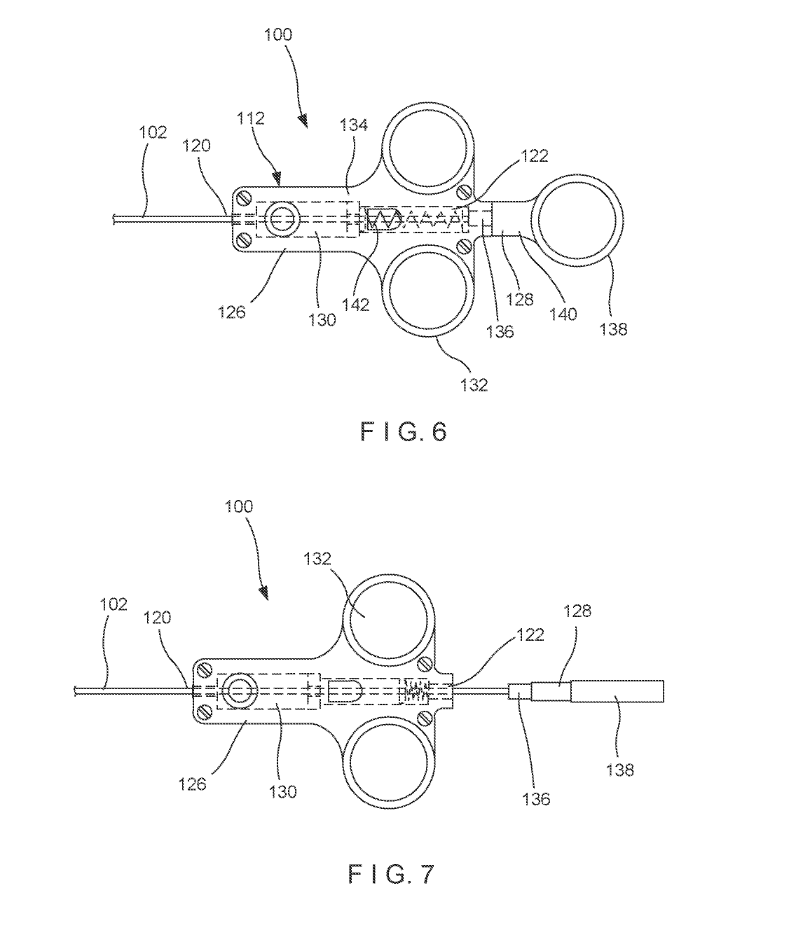

As shown in FIGS. 1-7, a biopsy device 100 according to an exemplary embodiment of the present disclosure comprises an outer sheath 102 and an inner needle 104 slidably received within a lumen 106 of the sheath 102 so that the needle 104 and the sheath 102 are movable relative to one another between an insertion/withdrawal configuration and a tissue collecting configuration. The needle 104 includes an opening or notch 110 extending laterally through a distal portion thereof for collecting a tissue sample therein. The device 100 further comprises a proximal portion including a handle assembly 112 which, when the sheath 102 and the needle 104 are inserted to a target tissue in a patient body via an incision in the skin, remains outside of the patient body. The handle assembly 112 is configured to move the sheath 102 and the needle 104 between the insertion/withdrawal configuration, in which the needle 104 is housed substantially within the sheath 102 so that the notch 110 is covered by a portion of the sheath 102, and the tissue collecting configuration, in which the notch 110 is distal of a distal end 118 of the sheath 102 to collect a tissue sample from the target tissue therethrough into the notch 110. The handle assembly 112 also includes a flushing port 114 and a collection port 116 connectable to, for example, a fluid flushing syringe and a collection vial, respectively, to permit the tissue sample collected in the notch 110 to be drawn back through the lumen 106 and into the handle assembly 112 so that the tissue sample may be flushed therefrom into the collection vial connected to the collection port 116 without removing the outer sheath 102 from the patient body. Thus, multiple tissue samples may be obtained using the device 100 without having to reinsert the device 100 into the patient body. This reduces the risk of infection and seeding of healthy tissue with unhealthy tissue.

The sheath 102 extends longitudinally from a proximal end 120 to the distal end 118 and includes the lumen 106 extending longitudinally therethrough from the proximal end 120 to the distal end 118. The distal end 118 may include a sharpened tapered tip to facilitate piercing of the target tissue. In one exemplary embodiment, the sheath 102 may extend along a substantially straight line. In another exemplary embodiment, the sheath 102 may extend along a curve so that the sheath 102 may extend into the target tissue off-axis and/or may be rotated about a longitudinal axis of the device 100 to collect tissue at different orientations. The sheath 102 may be formed of a metal material such as stainless steel or nitinol, which may be formed into a desired shape such as, for example, a curve.

The needle 104 extends longitudinally from a proximal end to a distal end 124, which may be tapered for piercing tissue as it is inserted therethrough. The notch 110 extends along a distal portion of the needle 104 so that when the needle 104 is advanced distally out of the sheath 102, the notch 110 is exposed to receive a tissue sample therein. Similarly to the outer sheath 102, the needle 104 may extend along a substantially straight line or may extend along a curve to be inserted into tissue off-axis and/or rotated to collect tissue from different orientations. The needle 104 may be formed of any suitable metal such as, for example, stainless steel or nitinol.

The needle 104 is sized and shaped to be slidably received within the lumen 106 of the outer sheath 102 so that the needle 104 may be moved between the insertion/withdrawal configuration and the tissue collecting configuration. In the insertion/withdrawal configuration, the needle 104 may be housed substantially within the outer sheath 102 so that the distal end 124 of the needle 104 does not extend distally past the distal end 118 of the outer sheath 102. In this insertion/withdrawal configuration, the sheath 102 extends over the needle 104 so that the notch 110 thereof is covered by an interior surface of the sheath 102 to prevent any tissue from being collected therein. In the tissue collecting configuration, the needle 104 is moved distally relative to the outer sheath 102 so that the distal end 124 of the needle 104 extends distally past the distal end 118 of the sheath 102 into the target tissue. Thus, a sample of the target tissue may be collected within the notch 110. Upon collection of the tissue sample within the notch 110, the device 100 is once again moved to the insertion/withdrawal configuration to capture the tissue sample therewithin. In particular, the sheath 102 may be advanced over the needle 104 so that the sharpened distal end 118 of the sheath 102 cuts the tissue sample from the surrounding tissue as it is being moved distally over the needle 104.

The handle assembly 112 further includes a first handle member 126 connected to the proximal end 120 of the sheath 102 and a second handle member 128 connected to the proximal end of the needle 104. The first and second handle members 126, 128 are movable relative to one another to move the sheath 102 and the needle 104 between the insertion/withdrawal configuration and the tissue collecting configuration. The first and second handle members 126, 128 are also releasably coupled to one another so that the second handle member 128 may be disengaged from the first handle member 126 to draw the needle 104 proximally relative to the outer sheath 102 from the insertion/withdrawal configuration to a sample retrieving configuration in which a tissue sample collected within the notch 110 may be flushed and collected within a collection vial or other tissue sample collecting reservoir via the ports 114, 116, as will be described in further detail below.

The first handle member 126 may include an elongated housing 130 including a handle lumen extending longitudinally therethrough and fingerloops 132 at a proximal end 134 thereof. Each of the first and second ports 114 include luer lock fittings, or any other connection features, for connecting the first and second ports 114, 116 to, for example, a fluid flushing syringe and collection vial, respectively. The first and second ports 114, 116 may extend from a portion of the housing 130 on, for example, diametrically opposing sides of the housing 130 so that, in the sample retrieving configuration, the second handle 128 may be moved proximally relative to the first handle 126 until the notch 110 of the needle 104 is aligned between the first and second ports 114, 116. The first and second ports 114 are open to the handle lumen 122 so that, when the notch 110 is positioned therebetween, fluid (e.g., saline) may be passed through the first port 114 and into the notch 110 to flush the tissue sample therefrom, through the second port 116 into the collection vial connected thereto. Although the first and second ports 114, 116 are described as being connectable to a fluid syringe and collection vial, respectively, it will be understood by those of skill in the art that the first and second ports 114, 116 may be connected to any of a variety of fluid flushing sources and tissue collection or storage elements.

The second handle member 128 may include a handle shaft 136 extending longitudinally and a fingerloop 138 at a proximal end 140 thereof, the fingerloop 138 may be particularly sized and shaped to receive a thumb of a user therein. The handle shaft 136 is sized and shaped to be slidably received within the handle lumen 122 so that the first and second handle members 126, 128 are movable between the insertion/withdrawal configuration and the tissue collecting configuration and between the insertion/withdrawal configuration and the sample retrieving configuration. In one exemplary embodiment, the handle assembly 112 may further include a spring 142 housed within the handle lumen 122 between portions of the housing 130 and the handle shaft 136 so that the device 100 may be “cocked” to facilitate a rapid firing of the device 100. The spring 142 biases the device 100 toward the insertion/withdrawal configuration so that when the second handle member 128 is drawn proximally relative to the first handle 126 (cocking the device 100), a tension is applied to the spring 142. Thus, when the device 100 is “fired” by pushing the second handle 128 distally with respect to the first handle 126 to move the device 100 from the insertion/withdrawal configuration to the tissue collecting configuration, the spring 142 is compressed and quickly reverts toward its biased configuration, moving the sheath 102 distally over the needle 104 so that the device 100 is once again in the insertion/withdrawal configuration. As discussed above, movement of the sheath 102 distally over the needle 104 cuts the tissue sample received within the notch 110 from a surrounding tissue while capturing the tissue sample therein.

The first and second handle members 126, 128 are also releasably coupled to one another so that, upon collection of a tissue sample within the notch 110, the first and second handle members 126, 128 may be disengaged from one another to permit the second handle member 128 to be drawn proximally out of the first handle member 126 until the device 100 is in the sample retrieving configuration. The handle lumen 122 and the handle shaft 136 may be keyed relative to one another so that, the second handle member 128 is rotatable about a longitudinal axis thereof relative to the first handle 126 (e.g., approximately 90 degrees) between a locked and unlocked position. In particular, in the cocking and firing positions described above, the handle assembly 112 is in the locked position. Rotation of the second handle 128 relative to the first handle 126 unlocks the handle assembly 112, permitting the second handle 128 to be withdrawn from the first handle 126. In one example, the handle lumen 122 and the handle shaft 136 may include a corresponding groove and pin configured to permit movement between the locked and unlocked positions.

According to an exemplary method for collecting a tissue sample using the device 100, a distal portion of the device 100 is inserted into a patient body in the locked insertion/withdrawal configuration. In particular, the sheath 102 and the needle 104 are inserted to a target tissue site within a patient body via an incision in the skin so that the handle assembly 112 remains outside of the patient body. Once inserted into the target tissue site, the device 100 may be cocked by drawing the second handle 128 proximally relative to the first handle 126. The device 100 is then fired by pushing the second handle 128 distally relative to the first handle 126 so that the device 100 is moved from the insertion/withdrawal configuration to the tissue collecting configuration. As the notch 110 is moved distally past the distal end 118 of the sheath 102, a first tissue sample is received within the notch 110. As described above, the spring 142 biases the device 100 toward the insertion/withdrawal configuration so that upon firing of the device 100, the spring 142 quickly reverts to its biased configuration, moving the sheath 102 distally over the needle 104 to capture the tissue sample within the notch 110.

Once the first tissue sample has been collected and captured within the notch 110, the second handle 128 may be rotated about the longitudinal axis relative to the first handle 126 to move the handle assembly 112 to the unlocked configuration. In the unlocked configuration, the second handle 128 may be withdrawn from the first handle 126 until the notch 110 of the needle 104 is aligned between the first and second ports 114, 116 of the first handle 126 so that the tissue sample may be flushed from the notch 110 via, for example, a fluid flushing syringe connected to the first port 114 and collected within, for example, a collection vial connected to the second port 116. The first tissue sample is flushed into the collection vial without removing the sheath 102 from the target tissue site. Thus, once the tissue sample has been flushed from the notch 110, the needle 104 may be reinserted into the sheath 102 and the handle assembly 112 once again moved to the locked configuration so that the device 100 may be cocked and fired to collect a second tissue sample. In embodiments in which the sheath 102 and/or the needle 104 extend along a curve, the device 100 may be rotated prior to firing the device 100 a second time, so that the second tissue sample may be collected from a different area of the target tissue. The first and second ports 114, 116 permit multiple samples to be collected by the device 100, without having to remove and insert the device 100 from the patient body, reducing the risk of infection and/or seeding of healthy tissue with unhealthy tissue.

It will be apparent to those skilled in the art that variations can be made in the structure and methodology of the present disclosure, without departing from the scope of the disclosure. Thus, it is intended that the present disclosure cover the modifications and variations of this disclosure provided that they come within the scope of the appended claims and their equivalents.

What is claimed is:

1. A device for acquiring a tissue sample, comprising:
an outer sheath extending longitudinally from a proximal end to a distal end and including a lumen extending therethrough;
an inner needle extending longitudinally from a proximal end to a distal end and sized and shaped to be slidably received within the lumen of the outer sheath to be moved between an insertion configuration and a tissue collecting configuration; and
a handle assembly connected to a proximal end of the outer sheath and including a first port configured to be connected to a fluid flushing source and a second port configured to be connected to a collection element so that, when the inner needle is drawn proximally relative to the outer sheath to the tissue collection configuration, the tissue sample is flushable into the collection element.

2. The device of claim 1, wherein the needle includes a notch extending laterally therein along a distal portion thereof so that, in the insertion configuration the notch is proximal of the distal end of the outer sheath and in the tissue collecting configuration the notch is distal of the distal end of the outer sheath

3. The device of claim 1, wherein the handle assembly includes a first handle member connected to the proximal end of the outer sheath and a second handle member connected to the proximal end of the inner needle, the first handle member including a handle lumen through which a portion of the second handle is slidably received.

4. The device of claim 3, wherein the first and second handle members are longitudinally movable relative to one another to move the device between the insertion configuration and the tissue collection configuration.

5. The device of claim 3, wherein the first and second ports are diametrically opposed from one another along a housing of the first handle member so that in the sample collection configuration the notch of the needle is aligned between the first and second ports.

6. The device of claim 3, wherein the first and second handle members are rotatable relative to one another about a longitudinal axis of the device to move the device between a locked configuration, in which the needle and the outer sheath are not movable relative to one another between the insertion and sample collection configurations, and an unlocked configuration in which the needle and the outer sheath are movable relative to one another between the insertion and sample collection configurations.

7. The device of claim 6, wherein the first and second handle members are keyed relative to one another and include a corresponding groove and pin.

8. The device of claim 1, wherein one of the inner sleeve and the outer sheath extends along a substantially straight line.

9. The device of claim 1, wherein one of the inner sleeve and the outer sheath extends along a curve.

10. The device of claim 1, wherein the handle assembly includes a spring biasing the device toward the insertion configuration.

11. A biopsy needle, comprising:
an outer sheath extending longitudinally from a proximal end to a distal end and including a lumen extending therethrough;
an inner needle extending longitudinally from a proximal end to a distal end and sized and shaped to be slidably received within the lumen of the outer sheath to be moved between an insertion configuration and a tissue collecting configuration, the needle including a notch extending laterally therein along a distal portion thereof, in the insertion configuration the notch is proximal of the distal end of the outer sheath and in the tissue collecting configuration the notch is distal of the distal end of the outer sheath; and
a handle assembly including a first handle member connected to a proximal end of the outer sheath and a second handle member connected to the proximal end of the inner needle, the first and second handle members longitudinally movable relative to one another to move the inner needle and the outer needle relative to one another between the insertion configuration and the tissue collection configuration, the first handle including a first port configured to be connected to a fluid flushing source and a second port configured to be connected to a tissue collection element so that, when the tissue sample is received within the notch and the inner needle is drawn proximally relative to the outer sheath to a sample collection configuration, the tissue sample is flushable from the notch into the tissue collection element.

12. The device of claim 11, wherein the handle assembly includes a spring element biasing the inner needle and outer sleeve toward the insertion configuration.

13. The device of claim 11, wherein the first and second handle members are rotatable relative to one another about a longitudinal axis of the device to move the device between a locked configuration, in which the needle and the outer sheath are not movable relative to one another between the insertion and sample collection configurations, and an unlocked configuration in which the needle and the outer sheath are movable relative to one another between the insertion and sample collection configurations.

14. The device of claim 11, wherein the first and second handle members are keyed relative to one another and include a corresponding groove and pin

15. The device of claim 11, wherein the first and second ports are diametrically opposed from one another along a housing of the first handle member so that in the sample collection configuration the notch of the needle is aligned between the first and second ports.

16. The device of claim 11, wherein one of the inner needle and the outer sheath extend along a curve.

17. A method for acquiring a tissue sample, comprising:
inserting a distal portion of a biopsy device to a target tissue site within a patient body via a skin incision, the distal portion of the biopsy device including an outer sheath and an inner needle slidably received therein, the inner needle including a notch extending laterally therein along a distal portion thereof;
moving the device from the insertion configuration, in which the notch of the inner needle is proximal of a distal end of the outer sheath, to a tissue collecting configuration in which the notch extends distally past the distal end of the outer sheath to collect a first tissue sample therein;
advancing the outer sheath distally over the notch of the inner needle back to the insertion configuration to capture the first tissue sample in the notch;
moving the inner needle proximally relative to the outer sheath until the notch is aligned between a first port and a second port of a handle assembly connected to a proximal end of the outer sheath; and
flushing the first tissue sample from the notch by passing a fluid through the first port into the notch and collecting the first tissue sample in a tissue storage element connected to the second port.

18. The method of claim 17, wherein the inner needle and the outer sheath are moved between the insertion configuration and the tissue collection configuration by moving a first handle member connected to the proximal end of the outer sheath and a second handle member connected to a proximal end of the inner needle longitudinally relative to one another.

19. The method of claim 18, wherein the inner needle and the outer sheath are moved from the insertion configuration to the sample collecting configuration by rotating the first and second handles relative to one another to unlock a longitudinal engagement between the first and second handles and permit the inner needle to be withdrawn relative to the outer sheath to the sample collection configuration.

20. The method of claim 17, wherein the outer sleeve remains in the target tissue during flushing and collecting of the first tissue sample, the method further comprising:
moving the device from the sample collection configuration to the insertion configuration; and
moving the device from the insertion configuration to the tissue collecting configuration to collect a second tissue sample in the notch; and
advancing the outer sheath distally over the notch of the inner needle back to the insertion configuration to capture the second tissue sample in the notch.
